SEQUENCE LISTING (6 sequences)

SEQ ID NO: 1 - DNA, 20 bp, Artificial Sequence
  Description of Artificial Sequence Synthetic primer
tccgctgccagtcgtcttcc

SEQ ID NO: 2 - DNA, 20 bp, Artificial Sequence
  Description of Artificial Sequence Synthetic primer
gtcctcgcgagtctaggcca

SEQ ID NO: 3 - DNA, 21 bp, Artificial Sequence
  Description of Artificial Sequence Synthetic primer
ggaactgcatccgttcatgag

SEQ ID NO: 4 - DNA, 20 bp, Artificial Sequence
  Description of Artificial Sequence Synthetic primer
tctttaaagcgttcgtggtc

SEQ ID NO: 5 - DNA, 19 bp, Artificial Sequence
  Description of Artificial Sequence Synthetic primer
actttcgatggtaggatag

SEQ ID NO: 6 - DNA, 20 bp, Artificial Sequence
  Description of Artificial Sequence Synthetic primer
tgatcgtcttcgatccccta

(19) United States
(12) Patent Application Publication
(10) Pub. No.: US 2007/0190545 A1
(43) Pub. Date: Aug. 16, 2007

(54) Multiplex PCR assay

(76) Inventors: Vishali Gupta, Chandigarh (IN); Naresh Sachdeva, Miami, FL (US); Amod Gupta, Chandigarh (IN); Sunil Arora, Chandigarh (IN); P. Bambery, Chandigarh (IN)

Correspondence Address: VENABLE LLP, P.O. BOX 34385, WASHINGTON, DC, 20043-9998, US

(21) Appl. No.: 11/598,069

(22) Filed: Nov. 13, 2006

(30) Foreign Application Priority Data
Nov. 14, 2005 (IN) 3045/DEL/2005

(51) Int. Cl. C12Q 1/68; C12P 19/34

(52) U.S. Cl. 435/6; 435/91.2

(57) ABSTRACT

This invention relates to a multiplex PCR assay capable of screening or detecting the relevant microbial organism specific to Mycobacterium tuberculosis, Toxoplasma gondii in a sample, comprising a reaction mixture of a combination of 3 sets of primers, one pair of said primers for detection of Mycobacterium tuberculosis, a second pair of said primers for detection of Toxoplasma gondii and a third pair of primers for the detection of pathogenically important fungi, said primers being compatible to each other.

### FIELD OF INVENTION

This invention relates to multiplex PCR assay capable of identifying a particular microbial organism in a sample.

### BACKGROUND OF THE INVENTION

Monoplex PCRs is a molecular technique for amplification of a selected gene fragment of the genome of any organism or cell using a specific set of primers specifically designed for that purpose. These primers can recognize and anneal (bind) to their predetermined (complimentary) sequence on the genome of that cell/organism. Then the reagents including the enzyme, buffers and the nucleotide mix (building blocks) are mixed together in proportion and put at temperature and conditions so that the enzyme can put the building blocks (nucleotides) in pre-specified sequence as per the complementarities to template (parent) strand of DNA.

Such monoplex PCR for diagnosing infections like Cytomegalo virus (CMV), Herpes simplex virus (HSV), Vericella zoster virus (VZV), Human Immunodeficiency virus (HIV), Toxoplasmosis gondii, Mycobacterium tuberculosis, Lyme disease and diseases like lymphomas and Whipple disease have been established. Thus, Monoplex PCR for any infection is known in the art. However, one major impediment to this technique is that one needs to perform a separate PCR reaction for each pathogen that could be time consuming and prohibitively expensive especially if one needs to test for a large number of potential pathogens. Also the monoplex examination would tax the available sample volume that might be very small in situation like intraocular samples.

Multiplex PCR assay is capable of screening various microbial organisms simultaneously or identify different alleles of one organism. In a multiplex PCR, a different cocktail of reagents is used for carrying all other ingredients of a reaction mix as in monoplex PCR situation in addition to the specifically designed primers for all the organisms which are to be identified or detected. However, in Multiplex PCR, the primers and the conditions that are applicable in a monoplex setting no longer produce same results because the primers for different organisms interfere with each other and reduce the sensitivity as well as specificity of assay. Thus both primer selection as well as optimization of conditions and concentration of reagents used need to be standardized, keeping in view the ‘nature’ of each and every primer as well as requirement of the sensitivity in that particular situation.

Dabil and coworkers have published earlier a technique of multiplex PCR assay, by using novel set of primers for a panel of common pathogens including CMV, HSV, VZV and Toxoplasma gondii (Ref: Dabil H, Boley M L, Schmitz T M and Van Gender R N. Validation of a diagnostic multiplex polymerase chain reaction assay for infectious posterior uveitis. Archieves of Ophthalmol 2001; 119:1315-22). Persson and Oslen have published Multiplex PCR reaction for identification of Campylobacter Coli and Campylobacter jejuni from pure cultures and directly on stool samples (Persson S and Oslen K E. J Med Microbiol. 2005; 54:1043-7).

### OBJECTS OF THE INVENTION

An object of this invention is to propose a multiplex PCR assay capable of identifying the relevant microbial organism in a sample.

Another object of this invention is to propose primers for Mycobacterium tuberculosis, Toxoplasma gondii and important fungi.

Further object of this invention is to propose a reaction mixture which can detect two or three organism in a sample at a time.

### SUMMARY OF THE INVENTION

According to this invention there is provided a multiplex PCR assay capable of screening or detecting the relevant microbial organism specific to Mycobacterium tuberculosis, Toxoplasma gondii in a sample, comprising a reaction mixture of a combination of 3 sets of primers, one pair of said primers for detection of Mycobacterium tuberculosis, a second pair of said primers for detection of Toxoplasma gondii and a third pair of primers for the detection of pathogenically important fungi, said primers being compatible to each other.

### DETAILED DESCRIPTION OF THE INVENTION

The present invention relates to multiplex PCR reactions for a triplex for Mycobacterium tuberculosis, Pathogenically important Fungi and Toxoplasmosis.

Primer Mycobacterium tuberculosis Having the Following Sequence:

| | |
|---|---|
| MPB1: | TCC GCT GCC AGT CGT CTT CC |
| MPB2: | GTC CTC GCG AGT CTA GGC CA |

For Toxoplasma gondii:

| | |
|---|---|
| B1F: | GGA ACT GCA TCC GTT CAT GAG |
| B1R: | TCT TTA AAG CGT TCG TGG TC |

For Pathogenically Important Fungi:

| | |
|---|---|
| B2F: | ACT TTC GAT GGT AGG ATA G |
| B4R: | TGA TCG TCT TCG ATC CCC TA |

a) Reagents Concentration:

Reaction Mixture (40 μl):

| | | |
|---|---|---|
| 10× Assay Buffer (0.1M | 4.0 | μl |
| Tris-HCL, PH 8.8, 15 mM MgCl₂, | | |
| 0.5M KCl and 1% Triton-X 100) | | |
| 25 mM MgCl₂ | 1.0 | μl (total 2.0 mM) |
| 10 mM dNTPs (each of A, T, G, C) | 1.5 | μl (375 μM) |
| 50 pmoles/μl pr-MpB1 | 0.5 | μl (0.625 pmoles/μl) |
| 50 pmoles/μl pr-MpB2 | 0.5 | μl (0.625 pmoles/μl) |
| 50 pmoles/μl pr-B1F | 0.5 | μl (0.625 pmoles/μl) |
| 50 pmoles/μl pr-B1R | 0.5 | μl (0.625 pmoles/μl) |
| 50 pmoles/μl pr-B2F | 0.5 | μl (0.625 pmoles/μl) |
| 50 pmoles/μl pr-B4R | 0.5 | μl (0.625 pmoles/μl) |
| 5 u/μl Taq DNA pol | 1.0 | μl (5 units) |
| Distilled water | 26.5 | μl |
| Template DNA | 1.0 | μl of each organism |

Thermo Cycling Conditions:

After initial denaturation at 94° C. for 3 min, thermo cycling was carried out for 35 cycles, with denaturation at 94° C. for 45 sec, annealing at 54° C. for 45 sec and extension at 72° C. also for 45 sec with last cycle of extension of 5 mins.

After amplification the PCR products were visualized on a 2.5% agarose gel following electrophoresis.

Reference to primers are:

| ORGANISM | PRIMERS | TARGET |
|---|---|---|
| M. tuberculosis | MPB1 and MPB2 | MPB64 gene |
| T. gondii | B1F and B1R | B1 gene |
| Fungi | B2F and B4R | 18s r DNA |

We claims:

1. A multiplex PCR assay capable of screening or detecting the relevant microbial organism specific to Mycobacterium tuberculosis, Toxoplasma gondii in a sample comprising a reaction mixture of a combination of 3 sets of primers, one of said primers for detection of Mycobacterium tuberculosis, a second of said primers for detection of Toxoplasma gondii and a third primer pair for the detection of pathogenically important fungi, said primers being compatible to each other.

2. The multiplex PCR assay as claimed in claim 1 wherein the primers for Mycobacterium tuberculosis are:

| | | |
|---|---|---|
| MPB1: | TCC GCT GCC AGT CGT CTT CC | (SEQ ID NO: 1) |
| MPB2: | GTC CTC GCG AGT CTA GGC CA | (SEQ ID NO: 2) |

for Toxoplasma gondii are:

| | | |
|---|---|---|
| B1F: | GGA ACT GCA TCC GTT CAT GAG | (SEQ ID NO: 3) |
| B1R: | TCT TTA AAG CGT TCG TGG TC | (SEQ ID NO: 4) |

for pathogenically important fungi:

| | | |
|---|---|---|
| B2F: | ACT TTC GAT GGT AGG ATA G | (SEQ ID NO: 5) |
| B4R: | TGA TCG TCT TCG ATC CCC TA | (SEQ ID NO: 6) |

3. The multiplex assay as claimed in claim 1 wherein said reaction mixture comprises:

| | | |
|---|---|---|
| 10× Assay Buffer (0.1M | 4.0 | μl |
| Tris-HCL, PH 8.8, 15 mM MgCl₂, | | |
| 0.5M KCl and 1% Triton-X 100) | | |
| 25 mM MgCl₂ | 1.0 | μl (total 2.0 mM) |
| 10 mM dNTPs (each of A, T, G, C) | 1.5 | μl (375 μM) |
| 50 pmoles/μl pr-MpB1 | 0.5 | μl (0.625 pmoles/μl) |
| 50 pmoles/μl pr-MpB2 | 0.5 | μl (0.625 pmoles/μl) |
| 50 pmoles/μl pr-B1F | 0.5 | μl (0.625 pmoles/μl) |
| 50 pmoles/μl pr-B1R | 0.5 | μl (0.625 pmoles/μl) |
| 50 pmoles/μl pr-B2F | 0.5 | μl (0.625 pmoles/μl) |
| 50 pmoles/μl pr-B4R | 0.5 | μl (0.625 pmoles/μl) |
| 5 U/μl Taq DNA pol | 1.0 | μl (5 units) |
| Distilled water | 26.5 | μl |
| Template DNA | 1.0 | μl of each organism |

4. A method for identifying the relevant microbial organism in a sample comprising the steps of:
preparing a mixture of primers compatible to each other;
treating the clinical sample with the said primers after initial denaturation thermo cycling being carried our for 35 cycles with denaturation, annealing at 54° C. for 45 sec and extension at 72° C. also for 45 sec with last cycle of extension of 5 mins
detecting the relevant micro-organism after amplification of the specific gene targets on 25% agarose gel.

5. A method as claimed in claim 4 wherein the primers for Mycobacterium tuberculosis are:

| | | |
|---|---|---|
| MPB1: | TCC GCT GCC AGT CGT CTT CC | (SEQ ID NO: 1) |
| MPB2: | GTC CTC GCG AGT CTA GGC CA | (SEQ ID NO: 2) |

for Toxoplasma gondii are:

| | | |
|---|---|---|
| B1F: | GGA ACT GCA TCC GTT CAT GAG | (SEQ ID NO: 3) |
| B1R: | TCT TTA AAG CGT TCG TGG TC | (SEQ ID NO: 4) |

for pathogenically important fungi:

| | | |
|---|---|---|
| B2F: | ACT TTC GAT GGT AGG ATA G | (SEQ ID NO: 5) |
| B4R: | TGA TCG TCT TCG ATC CCC TA | (SEQ ID NO: 6) |

6. A method as claimed in claim 4 wherein said reaction mixture comprises:—

| | | |
|---|---|---|
| 10× Assay Buffer (0.1M | 4.0 | μl |
| Tris-HCL, PH 8.8, 15 mM MgCl₂, | | |
| 0.5M KCl and 1% Triton-X 100) | | |
| 25 mM MgCl₂ | 1.0 | μl (total 2.0 mM) |
| 10 mM dNTPs (each of A, T, G, C) | 1.5 | μl (375 μM) |
| 50 pmoles/μl pr-MpB1 | 0.5 | μl (0.625 pmoles/μl) |
| 50 pmoles/μl pr-MpB2 | 0.5 | μl (0.625 pmoles/μl) |
| 50 pmoles/μl pr-B1F | 0.5 | μl (0.625 pmoles/μl) |
| 50 pmoles/μl pr-B1R | 0.5 | μl (0.625 pmoles/μl) |
| 50 pmoles/μl pr-B2F | 0.5 | μl (0.625 pmoles/μl) |
| 50 pmoles/μl pr-B4R | 0.5 | μl (0.625 pmoles/μl) |
| 5 U/μl Taq DNA pol | 1.0 | μl (5 units) |
| Distilled water | 26.5 | μl |
| Template DNA | 1.0 | μl of each organism |

7. A method as claimed in claim 4 wherein the initial denaturation is carried out at 94° C. for 3 minutes, said thermocycling with denaturation is carried our at 94° C. for 45 seconds.
